(19) United States
(12) Patent Application Publication
(10) Pub. No.: US 2006/0100448 A1
(43) Pub. Date: May 11, 2006

(54) Process for the production of Y-methyl-A-methylene-Y-butyrolactone from reaction of levulinic acid and hydrogen in the liquid phase followed by reaction of crude-Y-valerolactone and formaldehyde

(76) Inventors: Leo Ernest Manzer, Wilmington, DE (US); Keith W. Hutchenson, Lincoln University, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/270,128

(22) Filed: Nov. 9, 2005

Related U.S. Application Data
(60) Provisional application No. 60/626,630, filed on Nov. 10, 2004.

(51) Int. Cl. C07D 307/32

(52) U.S. Cl. 549/313

(57) ABSTRACT

An integrated two-step process for the production of gamma-methyl-alpha-methylene-gamma-butyrolactone from reaction of levulinic acid and hydrogen in the liquid phase to produce a crude GVL-containing product, followed by reaction of the crude GVL-containing product and formaldehyde in the supercritical or near-critical fluid phase.

### CROSS-REFERENCE TO RELATED APPLICATION

This application claims priority under 35 U.S.C. §119 from U.S. Provisional Application Serial No. 60/626,630, filed Nov. 10, 2004.

### FIELD OF THE INVENTION

The present invention relates to a method for making gamma-methyl-alpha-methylene-gamma-butyrolactone (MeMBL) using an integrated, two step process, the first step of which involves a reaction in which levulinic acid is reacted in the liquid phase with hydrogen in the presence of a catalyst to yield a crude, two phase reaction product, the liquid phase of which contains gamma-valerolactone (GVL), and the second step of which involves contacting the liquid phase, without separating unreacted levulinic acid therefrom, with formaldehyde and a solvent to form a reaction mixture that is in a supercritical or near-critical fluid phase in the presence of a catalyst to yield a reaction product that contains MeMBL.

### BACKGROUND

It is known that levulinic acid can be reacted in the liquid phase with hydrogen in the presence of a suitable catalyst to produce a product that contains gamma-valerolactone (hereinafter “GVL”). See U.S. Pat. No. 6,617,464 B2. It is also known that GVL can be reacted with formaldehyde in a supercritical or near-critical fluid phase in the presence of a suitable catalyst to produce a reaction product that contains gamma-methyl-alpha-methylene-gamma-butyrolactone (hereinafter “MeMBL”). See published U.S. application 2003/0166949 A1. Generally, the product of the levulinic acid conversion will be subjected to various purification procedures to produce a purified GVL, and the purified GVL will, in turn, be used as a reactant to produce MeMBL. It might be expected that the use of crude, i.e., unpurified, GVL as a reactant to make MeMBL would result in compromised yields of MeMBL, possibly because of the presence of trace impurities, such as, perhaps, residual acid, that could deactivate or otherwise adversely affect the catalyst used to convert the GVL into MeMBL.

### SUMMARY OF THE INVENTION

Surprisingly, it has now been found that the crude GVL-containing reaction product of the levulinic acid conversion reaction can be used directly, without separation of unreacted levulinic acid therefrom, as a reactant in the GVL to MeMBL conversion reaction, provided that the unreacted levulinic acid concentration does not exceed about 5 mol % relative to the combined GVL and levulinic acid.

More specifically, in its broadest aspect, the present invention is a method for making MeMBL, comprising:
(a) contacting in a first reactor levulinic acid and hydrogen, and, optionally, an optional organic liquid solvent having a higher vapor pressure than either levulinic acid or gamma-valerolactone (GVL), to form a first reaction mixture, at a temperature between 100° C. and 300° C. and a pressure between 0.45 and 21 MPa in the presence of a catalyst capable of converting the levulinic acid and hydrogen into gamma-valerolactone, for a period of time sufficient to achieve at least 95% conversion of levulinic acid, to form a first liquid reaction product that comprises GVL, unreacted hydrogen, any unreacted levulinic acid, and the optional organic liquid solvent, if one is used;
(b) separating the reaction product into a gas phase comprising a major portion of the unreacted hydrogen, and a first liquid phase comprising a major portion of the GVL, any unreacted levulinic acid, and a major portion of the optional organic liquid solvent, if one is used in step (a);
(c) separating the optional organic liquid solvent, if one is used in step (a), from the first liquid phase to produce a crude GVL-containing liquid phase; and
(d) introducing into a second reactor containing a second catalyst comprising a silica support and at least one element selected from the group consisting of potassium, cesium and rubidium, (i) either the first liquid phase, or the crude GVL-containing liquid phase if an optional organic liquid solvent is used in step (a), without any removal of the levulinic acid contained therein, (ii) a formaldehyde source capable of forming formaldehyde, and (iii) a solvent, to form in said second reactor a second reaction mixture at a temperature and pressure sufficient to cause the second reaction mixture to exist as a supercritical or near-critical fluid phase in contact with the second catalyst, thereby forming a second reaction product comprising MeMBL.

In a preferred embodiment, the invention further comprises introducing at at least a portion of the gas phase into the first reactor.

In another preferred embodiment the invention further comprises the steps of:
(e) decreasing the pressure of the second reaction product to cause the second reaction product to separate into (i) a second liquid phase comprising a major portion of the MeMBL, any unreacted GVL, and any unreacted formaldehyde, and (ii) a low density phase of lower density than said second liquid phase, said low density phase comprising the solvent; and
(f) separating the low density phase from the second liquid phase to produce a separated low density phase.

### BRIEF DESCRIPTION OF THE DRAWING

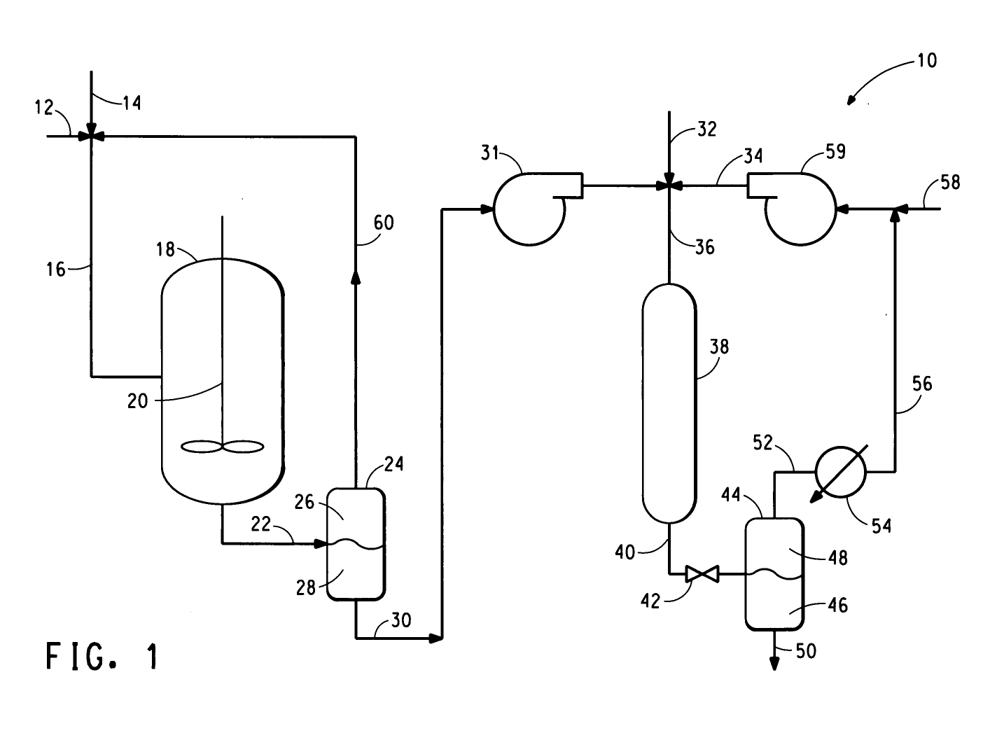

The Drawing consists of FIG. 1, which depicts a preferred embodiment of the present invention in schematic form.

### DETAILED DESCRIPTION OF THE INVENTION

Referring now to FIG. 1, there is shown in schematic form apparatus 10 for carrying out the process of the present invention as a continuous process. The present invention may also be carried out as a batch process, in which case references to “streams” should be interpreted
as fixed amounts of materials introduced or otherwise treated as a single batch.

An initial stream 12 of hydrogen is mixed with a stream 14 of levulinic acid and the combined stream 16 is introduced into first reactor 18 containing a catalyst (not shown) capable of converting levulinic acid (“LA”) to gamma-valerolactone (“GVL”). Alternatively, streams 12 and 14 may be introduced directly (not shown) into first reactor 18.

Suitable first reactors 18 include trickle bed, autoclave (batch or continuous stirred tank), and fixed bed reactors. The reactor should be configured to provide for adequate mixing of the hydrogen, levulinic acid and catalyst. One way to achieve this is to employ a mechanical agitator 20 in a suitable autoclave.

First reactor 18 contains a catalyst capable of converting the LA and hydrogen into a reaction product that contains GVL, any unreacted LA, and any unreacted hydrogen. First reactor 18 may also contain an optional nonreactive, organic liquid solvent (not shown) that has a vapor pressure higher than that of either levulinic acid or GVL, such as dioxane. The temperature and pressure conditions of reactor 18 should be in the range of 100° C. and 300° C. and between 0.45 and 21 MPa, to ensure a liquid phase reaction. Either or both of the levulinic acid 14 and hydrogen 12 can be preheated before being introduced into reactor 18. Similarly, the optional organic liquid solvent may also be preheated before being introduced into reactor 18. Alternatively, these components can be introduced into reactor 18 and heated and pressurized in situ. The temperature, pressure, catalyst loading and hold-up time should be selected to achieve at least 95% conversion of levulinic acid to GVL. One can determine the appropriate combination of conditions to achieve this level of conversion by running independent experiments in the reactor 18 that one chooses to employ in the method of the present invention.

Suitable catalysts for this first reaction include one or more elements selected from the group consisting of palladium, ruthenium, rhenium, rhodium, iridium, platinum, nickel, cobalt, copper, iron, and osmium. The catalytic element optionally can be supported on a support. The support can be in the form of powder, granules, pellets, or the like. A compound of the element also can be supported on a support. The depositing can be accomplished by a number of well-known methods. A preferred support material can be selected from the group consisting of carbon, alumina, silica, silica-alumina, silica-titania, titania, titania-alumina, barium sulfate, calcium carbonate, strontium carbonate, and various zeolites. Most preferred supports are alumina, titania and carbon. The catalysts of the present invention may optionally comprise catalyst additives and promoters that will enhance the efficiency of the catalyst. In the processes of the invention, the preferred catalytic element content range of the supported catalyst is from about 0.1% to about 20% of the supported catalyst based on catalyst weight plus the support weight. A more preferred catalytic element content range is from about 1% to about 10% of the supported catalyst. A further preferred catalytic element content range is from about 1% to about 5% of the supported catalyst.

Other suitable unsupported catalysts include the so-called Raney® catalysts. Raney® catalysts have a high surface area due to selectively leaching an alloy containing the active element(s) and a leachable element (usually aluminum). Raney® catalysts have high activity due to the higher specific area and allow the use of lower temperatures in hydrogenation reactions. The active elements of Raney® catalysts include nickel, copper, cobalt, iron, rhodium, ruthenium, rhenium, osmium, iridium, platinum, palladium, compounds thereof and combinations thereof. Promoter elements may also be added to the base Raney® elements (available from W.R. Grace & Co., Columbia Md.) listed above to affect selectivity and/or activity of the Raney® catalyst. Promoter elements for Raney® catalysts may be selected from transition elements from Groups 3 through Group 8, and Group 11 and Group 12 of the Periodic Table of the Elements. Examples of promoter elements for the Raney® based catalytic element include chromium, molybdenum, platinum, rhodium, ruthenium, osmium, and palladium, typically at about 2% by weight of the total element.

A stream 22 of first reaction product is withdrawn from first reactor 18 and introduced into separation device 24 in which two phases are formed: a gas phase 26 comprising a major portion of any unreacted hydrogen, and a first liquid phase 28 comprising a major portion of the GVL produced in first reactor 18. This first liquid phase also will contain any unreacted levulinic acid and a major portion of the optional organic liquid solvent, if one is used. Suitable separation devices 24 include a non-agitated vessel or tank that allows the reaction product to separate into two phases.

A stream 30 of first liquid phase 28 is withdrawn from separation device 24 using pump 31, and, without any removal of unreacted levulinic acid from the first liquid phase 28, stream 30 is combined with a stream 32 of a formaldehyde source capable of producing formaldehyde, a stream 34 of a solvent, and the combined stream 36 is introduced into second reactor 38 containing a second catalyst (not shown) capable of converting GVL into MeMBL.

In an alternative embodiment, not shown, in which the optional organic liquid solvent is used in first reactor 18, first liquid phase 28 can be treated, for example by distillation, to remove the optional organic liquid solvent contained therein to produce a crude GVL-containing liquid phase that can be introduced, as is stream 30, by pump 31 into second reactor 38.

A “formaldehyde source” is a material that is capable of forming formaldehyde under the conditions present for the reaction in the second reactor 38 of the present method, i.e., the conversion of GVL into MeMBL. Suitable formaldehyde sources include, but are not limited to, aqueous formalin, anhydrous formaldehyde, formaldehyde hemiacetal, a low molecular weight polyformaldehyde (paraformaldehyde), or formaldehyde trimer (trioxane). The use of paraformaldehyde, trioxane, or anhydrous formaldehyde is preferred since this reduces the need to remove water from the process. Hemiacetals work effectively, but require separate steps to release formaldehyde from the alcohol and to recover and recycle the alcohol.

Suitable second catalysts comprise a silica support and at least one element selected from the group consisting of potassium, rubidium, and cesium. The silica support optionally may be doped with aluminum, zirconium and/or titanium. These catalysts preferably contain from 0.1 to 40 wt % of the catalytic element relative to the combined weight of the support plus the element (as opposed to the compound of which the element is a part). Preferably the silica-supported catalyst is porous and has a pore size distribution such that pores having a diameter between 65 and 3200 Angstroms contribute a pore volume of at least 0.3 cubic centimeters per gram of catalyst. This requirement can be ascertained by using mercury or nitrogen porosimetry.

Typical factors to consider in selecting an appropriate solvent 34 for the second reaction mixture include solubility of reactants and products, chemical inertness, influence on the reaction rate and selectivity, cost, and toxicity. In addition, the critical temperature must be considered when selecting a potential solvent for conducting chemical transformations in the supercritical or near-critical fluid phase. For practical applications, thermal and catalytic chemical reactions can be conducted only in a relatively narrow temperature range. Lower temperatures result in unacceptable reaction rates, and higher temperatures can result in significant selectivity and yield losses, as well as catalyst deactivation. To obtain practical solvent densities and the corresponding density-dependent properties, temperature optimization must be balanced against a general desire to operate in the vicinity of the mixture critical point of the reaction system to fully exploit the potential advantages afforded by supercritical fluid (“SCF”) operation. The phase behavior of the reaction mixture, which is strongly influenced by the solvent critical temperature, is fundamentally important in defining this operating window, so one must select a solvent to provide the desired phase behavior. The phase behavior of SCF systems can also be manipulated to control the number and composition of coexisting phases, thus controlling both reaction effects as well as the separation of products or homogeneous catalysts from the reaction mixture. Finally, the addition of cosolvents can be effectively utilized to exploit specific solute interactions, such as enhancing solute solubilities and influencing reaction selectivities, and equilibria.

A reason often cited for using SCF-mediated reaction processes is the potential for utilizing a reaction medium that exhibits improved safety, health, and environmental impact relative to typical organic solvents. Carbon dioxide, in particular, is generally considered environmentally benign, nontoxic, nonflammable, and inexpensive, and it is suitable for use as a SCF solvent at relatively moderate temperatures. However, there are a variety of other practical SCF solvents that potentially have better solubility characteristics than CO₂ as well as beneficial impact relative to conventional liquid organic solvents.

Any suitable SCF solvent may be used in the process of this invention. A nonlimiting list of possible solvents include carbon dioxide, sulfur hexafluoride, fluoromethane, trifluoromethane, tetrafluromethane, ethane, ethylene, propane, propanol, isopropanol, propylene, butane, butanol, isobutane, isobutene, pentane, hexane, cyclohexane, benzene, toluene, o-xylene, water, and mixtures thereof, provided that it is inert to all reagents and products. Preferred SCF solvents include carbon dioxide or at least one C1 to C6 alkane, optionally substituted with Cl, F or Br.

The term “supercritical fluid” means a state of matter for a substance or a mixture of substances that exists above the critical temperature and critical pressure of the substance or mixture. For pure substances, the critical temperature and pressure are the highest at which vapor and liquid phases can coexist. Above the critical temperature, a liquid does not form for a pure substance, regardless of the applied pressure. Similarly, the critical pressure and critical molar volume are defined at this critical temperature corresponding to the state at which the vapor and liquid phases merge. Similarly, although more complex for multicomponent mixtures, the mixture critical state is identified as the condition at which the properties of coexisting vapor and liquid phases become indistinguishable. For a discussion of supercritical fluids, see Kirk-Othmer Encycl. of Chem. Technology, 4^{th} Ed., Vol. 23, pg. 452-477.

One of the primary advantages of SCF reaction media is that the density can be varied continuously from liquid-like to gas-like values by either varying the temperature or pressure, and to a first approximation, the solvent strength of the SCF media can be related to this continuously-variable solution density. The various density-dependent physical properties (e.g., solute solubility) also exhibit similar continuous variation in this region. In general, a SCF in the vicinity of its critical point has a liquid-like density and solvent strength, but exhibits transport properties (mass, momentum, and thermal diffusivities) that are intermediate to those of gases and liquids.

Since gaseous reactants are completely miscible with SCFs, their concentrations in SCF reaction media are significantly higher than are obtainable in conventional liquid solvents, even at appreciable pressures. These higher reactant concentrations in SCF media combined with increased component diffusivities and relatively low system viscosities can result in mass transfer rates that are appreciably higher than in liquid solvents. This can potentially shift a chemical reaction rate from mass transfer control to kinetic control in a reactor. The solubility of gaseous reactants in liquid solvents can also be enhanced by a volume expansion of the solvent with a dense supercritical fluid, which likewise results in increased mass transfer rates. Improved mass transport can also result in enhanced removal of residual solvents.

In practice, a number of desirable properties characteristic of the SCF state are realized in the expanded liquid region that exists at temperatures and pressures slightly below this critical point. A solution in this expanded liquid region is termed a “near-critical fluid” when the fluid is either at or below the critical temperature and the physical properties begin to approach those of a supercritical fluid. For the purposes of this invention, the term “near-critical fluid” includes those conditions where the fluid is at temperatures from about 75% of the critical temperature to about 100% of the critical temperature, and pressures from about 25% of the critical pressure to about 100% of the critical pressure.

The fluid state of the reaction mixture at any time during the course of a reaction is a function of the temperature, pressure, and composition. In practical applications of conducting reactions in the supercritical or near-critical fluid state, one selects an operating temperature, pressure, and mixture composition that corresponds to the desired supercritical or near-critical fluid state for the feed composition. This state may change during the course of the reaction as reactants are converted to products. For example, a reaction mixture in the SCF state may remain in the SCF state over the course of a reaction, or may undergo a phase transition to the near-critical fluid state. Conversely, a reaction mixture in the near-critical fluid state may remain in the near-critical fluid state over the course of a reaction, or may undergo a phase transition to the SCF state.

For practical applications of conducting chemical reactions in the supercritical or near-critical fluid state, one must determine the phase behavior of the mixture at the reaction conditions. One can visually observe the phase behavior of the reaction mixture by conducting the reaction in a vessel equipped with a transparent window, or by simulating the reaction mixture with a solution of similar concentration in such a vessel. Systematic determination of the phase boundaries of the reaction mixture can be determined by standard techniques using such a vessel that is also equipped with a means of varying the vessel volume at fixed composition and temperature. The vessel is loaded with the various components at the specified composition of the reaction mixture, heated to the reaction temperature. Then, the solution pressure is varied by changing the vessel volume until a phase transition is visually observed. After measuring the phase boundary of a solution of interest over the range of anticipated compositions, one can define the operating conditions (temperature and pressure) necessary to achieve the supercritical or near-critical state for conducting the desired reaction. For further discussion on experimentally determining fluid phase boundaries for a substance, see M. A. McHugh and V. J. Krukonis, Supercritical Fluid Extraction: Principles and Practice, 2^{nd} Ed., Butterworth-Heinemann: Boston (1994), pp. 85-98.

The temperature of the reaction mixture in second reactor 38 (the “second reaction mixture”) should be high enough to cause the chemical reaction to proceed with desired kinetics. In addition, the temperature and pressure of second reactor 38 must be chosen so that the second reaction mixture exists in a supercritical or near-critical fluid phase (or “state”) in contact with the second catalyst during the course of the reaction in second reactor 38. (See the discussion above for determining the phase behavior of the reaction mixture.) Under some reaction conditions, the second reaction mixture may transition between supercritical and near-critical fluid phases.

After a suitable contact time with the catalyst, a stream 40 of second reaction product is withdrawn from second reactor 38. The second reaction product contains MeMBL, any unreacted GVL and any unreacted formaldehyde, as well as any breakdown products of the formaldehyde source other than formaldehyde itself. The second reaction product will be in a supercritical or near-critical phase. The stream 40 is then introduced into a pressure-controlling device 42, in which the pressure of the second reaction product is reduced sufficiently to cause the second reaction product to separate in separation device 44 into two phases: (i) a second liquid phase 46 that contains a major portion of the MeMBL, as well as any unreacted GVL, and (ii) a “low density phase” 48 that is less dense than the second liquid phase, and contains a major portion of the solvent in stream 40. Suitable separation devices 44 include a non-agitated vessel or tank that allows the reaction product to separate into two phases.

The second liquid phase 46 may be withdrawn from the reactor as stream 50, which can be subjected to methods known in the art for separating MeMBL from unreacted GVL and formaldehyde source. A particularly suitable method for separating MeMBL from unreacted GVL involves polymerizing the MeMBL in the GVL solution using standard free radical polymerization, followed by precipitation of the poly-MeMBL, followed by thermal decomposition of the poly-MeMBL back to monomeric MeMBL. Another effective method is liquid/liquid extraction.

Optionally, a stream 52 of the low density phase is cooled in cooling device 54 to produce stream 56 of solvent that may be combined with a stream 58 of “make-up” solvent to produce stream 34 of solvent that may be introduced into second reactor 38 using pump 59.

Optionally, a stream 60 of gas phase 26, containing hydrogen, may be combined with streams 12 and 14 to produce stream 16, which is introduced into first reactor 18. Alternatively, stream 60 may be introduced directly into first reactor 18 (not shown).

The present process may be carried out in a batch mode, in which case the “streams” referred to above will refer to discrete quantities, or in a continuous mode.

### EXAMPLES

To simulate the effect of feeding unpurified product from the first reactor (containing varying amounts of unreacted levulinic acid) to the second reactor, a series of synthetic feed samples were prepared as follows and fed to a reactor (corresponding to second reactor 38) as described below. These experiments were conducted in a continuous fixed bed reactor consisting of a 0.38-inch o.d.×0.049-inch wall×11.5-inch long 316 stainless steel tube packed with catalyst. The reactor was heated by electrical band heaters mounted around an aluminum block enclosing the reactor. The reactor was charged with 2.0 g of 20% Rb/Engelhard KA-160 SiO₂ granular catalyst. The reactant feed solution consisted of about 38 wt % gamma-valerolactone, with varying relative concentrations of levulinic acid as noted in Table 1, 2.0 wt % diphenyl ether as an internal standard, with the balance being made up with an ethanol hemiacetal solution as the formaldehyde source. The ethanol hemiacetal was prepared by refluxing a 50 mol % paraformaldehyde solution in ethanol for four hours at 95° C. followed by cooling to room temperature and filtration. This solution resulted in a 2:1 ratio of formaldehyde to gamma-valerolactone in the reactor feed, which was metered at a rate resulting in a weight hour space velocity (WHSV) in the reactor of 0.65 g gamma-valerolactone/(g catalyst-h). CO₂ was used as the solvent, and the flow rate was metered independently to give a final total organic concentration (not including CO₂) of 5 mol % in the reactor feed. The reactor was operated at a temperature of 250° C. and a pressure of about 20 MPa. The corresponding reaction profiles showing conversion of gamma-valerolactone to MeMBL are summarized in Table 1.

TABLE 1

| | GVL | GVL | GVL | GVL | GVL |
|---|---|---|---|---|---|
| | Conv. % | Conv. % | Conv % | Conv % | Conv % |
| Run | with 0 | with 1 | with 2 | with 4 | with 8 |
| Time | mol % | mol % | mol % | mol % | mol % |
| (h) | LA* | LA* | LA* | LA* | LA* |
| 0.9 | — | — | 76.3 | — | — |
| 1.0 | — | — | — | 79.5 | 58.0 |
| 1.1 | 85.7 | — | 75.6 | — | — |
| 1.3 | 84.3 | — | 76.0 | 76.8 | 57.3 |
| 1.5 | 85.0 | — | 76.0 | 76.4 | 55.4 |
| 1.7 | — | 72.5 | 76.4 | 76.7 | 51.7 |
| 2.0 | 87.9 | 73.6 | 76.4 | 76.7 | 44.6 |
| 2.3 | 90.0 | 74.1 | 76.0 | 75.8 | — |

*Molar LA concentration is relative to total GVL plus LA.

The data in Table 1 show that although there may be a modest deactivation of the catalyst used to convert the GVL to MeMBL if levulinic acid is present in the reaction mixture, the deactivation is independent of the levulinic acid concentration up to about 5 mol % relative to the combined GVL and levulinic acid. Little variation in GVL conversion is observed for residual LA feed compositions of 1, 2, and 4 mol %. However, at a LA feed composition of 8 mol %, significantly reduced GVL conversion is observed, indicating unacceptably reduced catalyst activity. Therefore, if the crude GVL-containing reaction product of the levulinic acid conversion reaction contains no more than about 5 mol % unreacted levulinic acid, then the crude GVL-containing reaction product can be used directly, without separation of unreacted levulinic acid therefrom, as a reactant in the GVL to MeMBL conversion reaction. Although the data in Table 1 show the effect of levulinic acid on the catalytic activity of a rubidium-based catalyst, based on the similar catalytic activity of cesium, potassium, and rubidium for the conversion of GVL to MeMBL, as disclosed in U.S. 2003/0166949 A1, a similar result should be achievable with cesium and potassium-based catalysts in the present invention.

1. A method for making gamma-methyl-alpha-methylene-gamma-butyrolactone (MeMBL), comprising:
(a) contacting in a first reactor levulinic acid and hydrogen, and, optionally, an optional organic liquid solvent having a higher vapor pressure than either levulinic acid or gamma-valerolactone (GVL), to form a first reaction mixture, at a temperature between 100° C. and 300° C. and a pressure between 0.45 and 21 MPa in the presence of a catalyst capable of converting the levulinic acid and hydrogen into gamma-valerolactone, for a period of time sufficient to achieve at least 95% conversion of levulinic acid, to form a first liquid reaction product that comprises GVL, unreacted hydrogen, any unreacted levulinic acid, and the optional organic liquid solvent, if one is used;
(b) separating the reaction product into a gas phase comprising a major portion of the unreacted hydrogen, and a first liquid phase comprising a major portion of the GVL, any unreacted levulinic acid, and a major portion of the optional organic liquid solvent, if one is used in step (a);
(c) separating the optional organic liquid solvent, if one is used in step (a), from the first liquid phase to produce a crude GVL-containing liquid phase; and
(d) introducing into a second reactor containing a second catalyst comprising a silica support and at least one element selected from the group consisting of potassium, cesium and rubidium, (i) either the first liquid phase, or the crude GVL-containing liquid phase if an optional organic liquid solvent is used in step (a), without any removal of the levulinic acid contained therein, (ii) a formaldehyde source capable of forming formaldehyde, and (iii) a solvent, to form in said second reactor a second reaction mixture at a temperature and pressure sufficient to cause the second reaction mixture to exist as a supercritical or near-critical fluid phase in contact with the second catalyst, thereby forming a second reaction product comprising MeMBL.

2. The process of claim 1 further comprising introducing at least a portion of the gas phase into the first reactor.

3. The process of claim 1 further comprising the steps of:
(e) decreasing the pressure of the second reaction product to cause the second reaction product to separate into (i) a second liquid phase comprising a major portion of the MeMBL, any unreacted GVL, and any unreacted formaldehyde, and (ii) a low density phase of lower density than said second liquid phase, said low density phase comprising the solvent; and
(f) separating the low density phase from the second liquid phase to produce a separated second phase.

4. The process of claim 3 further comprising introducing at least a portion of the separated low density phase into the second reactor.

5. The process of claim 4 further comprising introducing at least a portion of the gas phase into the first reactor.

6. The process of claim 1 wherein the solvent is selected from the group consisting of carbon dioxide and at least one C1 to C6 alkane, optionally substituted with Cl, F, or Br.
